(19) United States
(12) Patent Application Publication
(10) Pub. No.: US 2022/0370027 A1
(43) Pub. Date: Nov. 24, 2022

(54) X-RAY INSPECTION APPARATUS

(71) Applicant: ANRITSU CORPORATION, Kanagawa (JP)

(72) Inventors: Naoya SAITO, Kanagawa (JP); Akihiro NODA, Kanagawa (JP); Keishi OKANO, Kanagawa (JP)

(21) Appl. No.: 17/663,874

(22) Filed: May 18, 2022

(30) Foreign Application Priority Data
May 21, 2021 (JP) 2021-086199

(51) Int. Cl. A61B 6/00

(52) U.S. Cl. CPC A61B 6/4488; A61B 2562/0271; A61B 2562/029

(57) ABSTRACT

There is provided an X-ray inspection apparatus that inspects an inspection object by irradiating the inspection object with X-rays and detecting transmitted X-rays, the apparatus includes a module group (X-ray detection portion) which is disposed inside a housing and in which an electric component that is vulnerable to dew condensation caused by cooling of cooling means is mounted, a humidity sensor that monitors a humidity inside the housing, dehumidifying means (air conditioner) for dehumidifying an inside of the housing, and a control portion that performs supplying of power to the module group when the humidity monitored by the humidity sensor is equal to or lower than a predetermined value.

### TECHNICAL FIELD

The present invention relates to an X-ray inspection apparatus.

### BACKGROUND ART

In the related art, an X-ray inspection apparatus is used to detect foreign substances (metals, bones, glass, stones, synthetic resin materials, and the like) in various types of inspection objects (including surfaces) such as raw meat, fish, processed foods, and pharmaceuticals, for example. In the X-ray inspection apparatus, heat is generated by driving various electronic components such as an X-ray source or an X-ray detector. When these heats are trapped inside a housing, a temperature may exceed a specification temperature of the electronic component (for example, 40° C.), so it is necessary to cool the inside of the housing and to reduce the temperature. In the X-ray inspection apparatus disclosed in Patent Document 1, an air conditioner is attached to a back surface of the housing, and the inside of the housing is cooled by a cooling portion of the air conditioner.

### RELATED ART DOCUMENT

### [Patent Document]

[Patent Document 1] JP-A-2018-155550

### DISCLOSURE OF THE INVENTION

### Problem that the Invention is to Solve

The X-ray inspection apparatus has various configurations for cooling the X-ray source, the X-ray detector, and the like, and may include the air conditioner as described above. By providing the air conditioner, it is not necessary to take in outside air, and it is possible to suppress an increase in temperature due to heat generated from an electric unit constituting the X-ray inspection apparatus, while maintaining waterproofness or dustproofness in the used environment. However, in a case of using the X-ray inspection apparatus in a high-humidity environment, a cover that is normally closed may be opened due to maintenance work and the like, so high-humidity outside air is taken into the apparatus and the cooling is performed by the air conditioner as it is. Then, water droplets adhere to a surface of an electric component constituting the electric unit inside the apparatus, for example, a semiconductor chip, which causes a failure.

The present invention has been made in view of the above circumstances, and an object of the present invention is to provide an X-ray inspection apparatus capable of suppressing a failure of an electric component, by preventing local dew condensation on a surface and surrounding of the electric component that may occur due to a temperature drop caused by cooling means for cooling an inside of the apparatus such as an air conditioner.

### Means for Solving the Problem

Next, the means for solving the above problems will be described with reference to the drawings corresponding to an embodiment.

According to a first aspect of the present invention, there is provided an X-ray inspection apparatus that inspects an inspection object by irradiating the inspection object with X-rays and detecting transmitted X-rays, the apparatus including: a housing; cooling means for cooling an inside of the housing; a module group which is disposed inside the housing and in which an electric component that is vulnerable to dew condensation caused by cooling of the cooling means is mounted; a humidity sensor that monitors a humidity inside the housing; and a control portion that performs supplying of power to the module group when the humidity monitored by the humidity sensor is equal to or lower than a predetermined value.

In this X-ray inspection apparatus 1, the humidity inside a housing 4 is monitored by a humidity sensor 15. The control portion performs supplying of power to each electric component, based on a detected value of the humidity sensor 15. Even in a state in which the main power supply is turned on, if the humidity by the humidity sensor 15 is equal to or higher than the predetermined value, an X-ray detection portion 6 including the module group is not turned on. As a result, the cooling means such as a Pelche element provided in the module group in the X-ray detection portion 6 is not driven in a humid state, so that it is possible to suppress a failure of the module group due to adhesion of water droplets due to dew condensation at a semiconductor chip. When the detected value of the humidity sensor 15 is equal to or lower than the predetermined value, the control portion performs supplying of power to the module group, that is, the X-ray detection portion 6. Therefore, in the X-ray inspection apparatus 1, for example, at a start of an operation, it is possible to effectively suppress dew condensation of the module group due to the X-ray detection portion 6 being driven while high-humidity outside air is taken into the housing 4. As a result, with the X-ray inspection apparatus 1, it is possible to prevent dew condensation of local electric components caused by the cooling means such as an air conditioner 9, and suppress a failure of the electric component.

According to the X-ray inspection apparatus of the first aspect, in the X-ray inspection apparatus according to a second aspect of the present invention, after the X-ray inspection apparatus is activated, the control portion starts the supplying of power to the module group, under a condition that the humidity monitored by the humidity sensor is equal to or lower than the predetermined value.

When the main power supply of the X-ray inspection apparatus 1 is turned on and a warm-up operation is completed, first, a control portion 16 turns on the humidity sensor 15 and then monitors the value which is detected by the humidity sensor 15 in a state in which only the power supplied to the X-ray detection portion 6 as the module group is turned off. In a case where the detected value is monitored and the detected value of the humidity sensor 15 falls below the threshold value, the control portion 16 turns on the power supply to the X-ray detection portion 6. That is, after checking that the humidity is not high, the power of the module group that is vulnerable to dew condensation is turned on. As a result, the X-ray inspection apparatus 1 can solve the problem that dew condensation occurs at the module group, at the start of the operation. After that, the operation as the X-ray inspection apparatus, that is, an inspection will start.

According to the X-ray inspection apparatus of the first aspect, the X-ray inspection apparatus according to a third aspect of the present invention, further includes: dehumidifying means for dehumidifying the inside of the housing, in which when the humidity monitored by the humidity sensor is equal to or higher than the predetermined value, the control portion causes the dehumidifying means to dehumidify the inside of the housing, and stops the supplying of power to the module group.

In addition, according to the X-ray inspection apparatus of the second aspect, the X-ray inspection apparatus according to a fourth aspect of the present invention, further includes: dehumidifying means for dehumidifying the inside of the housing, in which when the humidity monitored by the humidity sensor is equal to or higher than the predetermined value, the control portion causes the dehumidifying means to dehumidify the inside of the housing, and stops the supplying of power to the module group.

As a result, it is possible to prevent the X-ray inspection apparatus 1 from being operated in a state in which the inside of the housing is highly humid at the start of the operation and during the operation of the X-ray inspection apparatus 1.

According to the X-ray inspection apparatus of the third aspect, in the X-ray inspection apparatus according to a fifth aspect of the present invention, in which the control portion further issues an alarm when the humidity monitored by the humidity sensor is equal to or higher than the predetermined value.

In addition, according to the X-ray inspection apparatus of the fourth aspect, in the X-ray inspection apparatus according to a sixth aspect of the present invention, in which the control portion further issues an alarm when the humidity monitored by the humidity sensor is equal to or higher than the predetermined value.

As a result, it is possible to notify an operator that the inside of the housing is in a high humidity state.

According to the X-ray inspection apparatus of the third aspect, in the X-ray inspection apparatus according to a seventh aspect of the present invention, the cooling means includes an air conditioner, and the air conditioner has a dehumidifying function as the dehumidifying means.

In addition, according to the X-ray inspection apparatus of the fourth aspect, in the X-ray inspection apparatus according to an eighth aspect of the present invention, the cooling means includes an air conditioner, and the air conditioner has a dehumidifying function as the dehumidifying means.

In this X-ray inspection apparatus 1, the cooling means includes the air conditioner 9. The air conditioner 9 has the dehumidifying function. The purpose of dehumidification is to remove moisture, and cold air is blown out from the evaporator in the same manner as cooling. Further, in the X-ray inspection apparatus 1, the X-ray generation source is desired to be warmed up to a predetermined temperature so as to stably irradiate X-rays. Meanwhile, the X-ray generation source is not desired to be heated up to a high temperature. Further, the module group having the semiconductor chip is warmed by self-heating, and from the viewpoint of noise reduction and life, there are different demands for each electric unit built in the housing 4, such as cooling to a predetermined temperature. Therefore, the operating temperature of each electric unit inside is stabilized by the warm-up operation at the start of the operation, and the air conditioner 9 performs not only the dehumidifying operation but also the cooling operation. As a result, by using the function of the air conditioner 9, the X-ray inspection apparatus 1 can switch between cooling the inside of the housing 4 and dehumidifying the inside of the housing 4, and an atmosphere of the module group can be made less likely to cause dew condensation.

According to the X-ray inspection apparatus of the seventh aspect, in the X-ray inspection apparatus according to a ninth aspect of the present invention, inside the housing, a non-condensation module group that is not affected by dew condensation and different from the module group is provided, and a temperature sensor that monitors a temperature inside the housing is provided, and the control portion supplies power to the non-condensation module group, and then starts an operation of a cooling function by the air conditioner in a case where the temperature monitored by the temperature sensor is equal to or higher than a predetermined value.

According to the X-ray inspection apparatus of the eighth aspect, in the X-ray inspection apparatus according to a tenth aspect of the present invention, inside the housing, a non-condensation module group that is not affected by dew condensation and different from the module group is provided, and a temperature sensor that monitors a temperature inside the housing is provided, and the control portion supplies power to the non-condensation module group, and then starts an operation of a cooling function by the air conditioner in a case where the temperature monitored by the temperature sensor is equal to or higher than a predetermined value.

With this X-ray inspection apparatus 1, in a case where the temperature detected by the temperature sensor becomes equal to or higher than the predetermined value, the operation of the cooling function by the air conditioner 9 is started. That is, for example, the X-ray generation source 5 or the like, which is a non-condensation module group, is heated by energization, and then the cooling function or the dehumidifying function of the air conditioner 9 is turned on. As a result, it becomes possible to maintain the temperature inside the housing 4 stabilized by the warm-up operation, a performance change of the electric unit due to a temperature change can be suppressed, and the performance of the X-ray inspection apparatus is stabilized. The X-ray generation source 5 has a tank, and the tank is filled with cooling oil to accommodate the X-ray source. A tank cooling portion provided on an upper surface of the tank has a heat dissipation fin covered with a cover. At a start of the operation, the X-ray inspection apparatus 1 requires the warm-up operation so as to bring the X-ray source to a predetermined temperature. However, in order to avoid becoming too warm, that is, a high-temperature state, cooling by the air conditioner 9 is required. As a result, the warm-up operation can be performed and the inside of the housing 4 can be dehumidified.

According to the X-ray inspection apparatus of the ninth aspect, in the X-ray inspection apparatus according to an eleventh aspect of the present invention, the control portion switches between a cooling operation and a dehumidifying operation of the air conditioner, based on a detected value of the temperature sensor and a detected value of the humidity sensor.

In addition, according to the X-ray inspection apparatus of the tenth aspect, in the X-ray inspection apparatus according to a twelfth aspect of the present invention, the control portion switches between a cooling operation and a dehumidifying operation of the air conditioner, based on a detected value of the temperature sensor and a detected value of the humidity sensor.

As a result, it is possible to efficiently operate the X-ray inspection apparatus 1.

### Advantage of the Invention

With the X-ray inspection apparatus according to claim 1 of the present invention, it is possible to prevent dew condensation of the module group, in which the electric component that is vulnerable to dew condensation is mounted, due to cooling of the cooling means, and to suppress a failure of the electric component.

With the X-ray inspection apparatus according to claim 2 of the present invention, it is possible to solve the problem that dew condensation occurs at the module group, at the start of the operation.

With the X-ray inspection apparatus according to claim 3 or 4 of the present invention, it is possible to prevent the X-ray inspection apparatus 1 from being operated in a state in which the inside of the housing is highly humid at the start of the operation and during the operation of the X-ray inspection apparatus 1.

With the X-ray inspection apparatus according to claim 5 or 6 of the present invention, it is possible to notify the operator that the inside of the housing is in a high humidity state.

With the X-ray inspection apparatus according to claim 7 or 8 of the present invention, by using the function of the air conditioner, it is possible to switch between cooling the inside of the housing and dehumidifying the inside of the housing, and an atmosphere of the module group can be made less likely to cause dew condensation.

With the X-ray inspection apparatus according to claim 9 or 10 of the present invention, the warm-up operation can be performed and the inside of the housing can be dehumidified.

With the X-ray inspection apparatus according to claim 11 or 12 of the present invention, it is possible to efficiently operate the X-ray inspection apparatus.

### BRIEF DESCRIPTION OF THE DRAWINGS

FIG. 1 is a side view of a housing portion of an X-ray inspection apparatus according to an embodiment.

### BEST MODE FOR CARRYING OUT THE INVENTION

Hereinafter, an embodiment according to the present invention will be described with reference to the drawings.

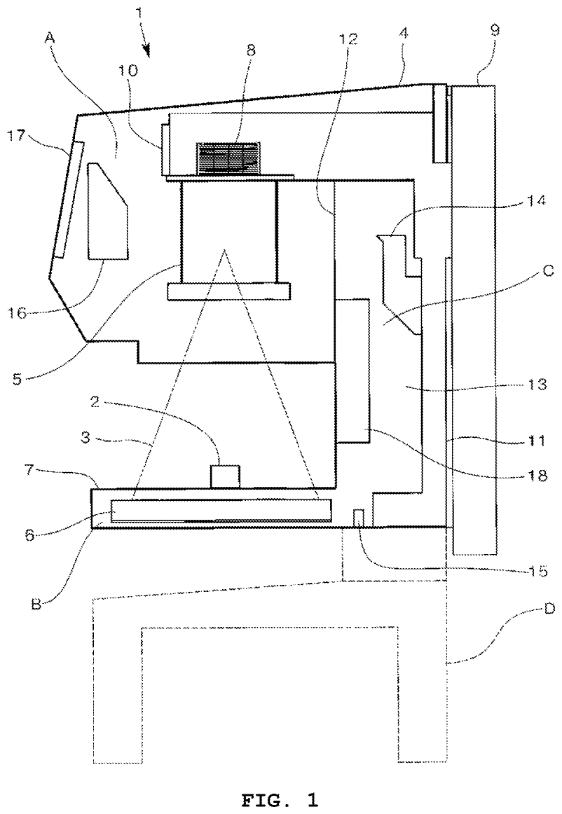

FIG. 1 is a side view of an X-ray inspection apparatus 1 according to the embodiment.

The X-ray inspection apparatus 1 according to the present embodiment inspects an inspection object 2 by irradiating the inspection object 2 with X-rays and detecting transmitted X-rays 3. As the inspection object 2, for example, raw meat, fish, processed foods, pharmaceuticals, and the like are used.

The X-ray inspection apparatus 1 includes an X-ray generation source 5 that generates the X-rays 3, an X-ray detection portion 6 that receives the X-rays 3 from the X-ray generation source 5, and transport means 7 that transports the inspection object 2 between the X-ray generation source 5 and the X-ray detection portion 6, in a housing 4. When the X-ray inspection apparatus 1 is used in an inspection step of a production line for foods, chemicals, and the like, for example, the inspection object 2 is sequentially conveyed from an upstream manufacturing step or processing step, and a predetermined inspection is repeated. The inspection result is displayed on an operation panel 17 and a signal is output to another apparatus. The inspected inspection object 2 is sequentially conveyed out to a downstream sorting step or packaging step.

In the present embodiment, the housing 4 is configured with an upper housing A that accommodates the X-ray generation source 5, a lower housing B that accommodates the X-ray detection portion 6, and a back housing C that connects the upper housing A and the lower housing B on a back side of the X-ray inspection apparatus 1 so as to form an irradiation region for irradiating the inspection object 2 with the X-rays 3, and is supported by a leg portion D.

The X-ray generation source 5 emits the X-ray 3 on the inspection object 2, which is transported by the transport means 7 provided with a conveyor belt that circulates and moves. The X-ray generation source 5 has a configuration in which a tank is provided, and the tank is filled with cooling oil to accommodate an X-ray tube serving as an X-ray source. A tank cooling portion provided on an upper surface of the tank has a heat dissipation fin 8 covered with a cover. The X-ray generation source 5 is disposed in the upper housing A of the housing 4, and irradiates the X-rays 3 downward. The X-ray 3 is irradiated in a substantially conical shape extending downward from the X-ray tube, and then becomes a planar shape extending downward via an elongated hole formed in a bottom portion of the X-ray generation source 5. In the X-ray generation source 5, heat generated when the X-ray 3 is generated is dissipated by a heat dissipation fin 8.

The X-ray detection portion 6 detects the X-ray 3 transmitting the inspection object 2, which is disposed in the lower housing B of the housing 4 and below the conveyor belt. The X-ray detection portion 6 has a slit and passes the planar X-ray 3 emitted from the X-ray generation source 5. The X-ray 3 passing through the slit is received by a sensor portion of the X-ray detection portion 6. In the sensor portion, a semiconductor chip, which is a detection element, is locally cooled by a Pelche element. The X-ray detection portion 6 photoconverts the X-ray 3, and outputs the resultant X-ray to an X-ray processing portion (not illustrated).

The housing 4 is provided with an air conditioner 9 as cooling means at a back portion. The air conditioner 9 has a refrigeration cycle. That is, the air conditioner 9 has a cooling function of cooling an inside of the housing 4 by passing air circulating inside the housing 4 through an evaporator, and a dehumidifying function of draining dew water generated in the evaporator and dehumidifying the inside of the housing 4 by passing weak air through the evaporator.

The air conditioner 9 has a built-in temperature sensor, and includes a controller that turns on a cooling operation when an operation start temperature on a high-temperature side is reached and turns off the cooling operation when a set temperature on a low-temperature side is reached, based on an output of this temperature sensor. This controller can also switch between the cooling operation and the dehumidifying operation of the air conditioner 9.

As the air conditioner 9, an air conditioner to which a high or low cooling performance is set based on a maximum increase in temperature determined by a volume of a space to be cooled or the amount of heat generated in the space, or an air conditioner having a predetermined cooling performance is selected. In particular, in a case where the amount of heat generated by the X-ray generation source 5 is large and high cooling performance is required, the large air conditioner 9 is attached to the back housing C of the housing 4.

The housing 4 includes an upper branch duct 10 that supplies air (cold air) from an air outlet of the air conditioner 9 to the X-ray generation source 5 of the upper housing A and a lower branch duct 11 that supplies the air to the X-ray detection portion 6 of the lower housing B. The air supplied from the air conditioner 9 to the X-ray generation source 5 and the X-ray detection portion 6 is returned from a suction duct 14 disposed in a return chamber 13 defined by a partition plate 12 partitioning the inside of the housing 4 to a suction port of the air conditioner 9 and circulates inside the housing 4. As a result, both the X-ray generation source 5 and the X-ray detection portion 6 are cooled.

A plurality of various electric components are provided as electric units inside the housing 4. Among them, there is a group of modules in which electric components having low durability against adhesion of water droplets, that is, vulnerable to dew condensation are mounted. An example of this module group includes a line sensor unit provided with a semiconductor chip adopted in the X-ray detection portion 6, and the like. The line sensor unit is vulnerable to heat, that is, an allowable range of operating temperatures is narrow, and the maximum operating temperature that defines an upper limit temperature during operation is approximately 20° C. to 30° C. lower than other electric units. Therefore, a Pelche element as cooling means for the line sensor unit is provided in contact with the semiconductor chip so as to locally exhaust heat from the semiconductor chip mainly by heat conduction, suppress the increase in temperature, and protect the line sensor unit from the heat. As such local cooling means, cooling means to which a high or low cooling performance is set based on the amount of heat and the operating temperature range of the electric component to be cooled, or cooling means having a predetermined cooling performance is selected. For example, when small cooling means such as a Pelche element is selected, a low-temperature surface of the Pelche element is brought into contact with the electric component side, heat is absorbed from the contact surface, and the heat is transferred to a high-temperature surface. The increase in temperature on the high-temperature surface is diffused inside the X-ray detection portion 6 by circulation of air by the air conditioner 9, and the heat is dissipated.

The X-ray inspection apparatus 1 is provided with a humidity sensor 15 that monitors a humidity inside the housing 4. The humidity sensor 15 may be disposed in the group of the modules in which the electric components vulnerable to dew condensation are mounted, that is, in the lower housing B or a lower portion of the back housing C of the housing 4 in the vicinity of the X-ray detection portion 6, which is a space in which the temperature drops due to the cooling of the Pelche element described above, which is local cooling means. As a result, it is possible to accurately detect a high humidity state in which dew condensation that causes a failure of the X-ray detection portion 6 is likely to occur.

The X-ray inspection apparatus 1 includes dehumidifying means for dehumidifying the inside of the housing 4. In the present embodiment, this dehumidifying means is realized by a dehumidifying operation of the air conditioner 9 described above. As the dehumidifying means, a dehumidifier dedicated to dehumidification may be used instead of the dehumidifying operation of the air conditioner 9.

The X-ray inspection apparatus 1 includes a first control portion 16. The first control portion 16 includes a central processing unit (CPU), a storage portion, connection ports or communication interfaces for various sensors, and the like. The first control portion 16 controls to supply power to the module group when a humidity detected by the humidity sensor 15 is equal to or lower than a predetermined value, or controls to stop the supplying of power to the module group when the detected humidity is equal to or higher than the predetermined value. The operation panel 17 provided on a front surface of the housing 4 is connected to the first control portion 16.

In the housing 4, apart from the module group vulnerable to dew condensation, a module group (referred to as a non-condensation module group) that is not affected by dew condensation, that is, has a structure in which a failure is less likely to occur due to dew condensation or dew condensation itself is less likely to occur is provided. As the non-condensation module group, for example, the X-ray generation source 5 in which electric components are housed in a metal case filled with insulating oil, and which also serves as a heat generation source is used. Further, the X-ray inspection apparatus 1 includes a temperature sensor that monitors a temperature inside the housing 4. This temperature sensor may be provided at a plurality of positions, for example, such as in the vicinity of a unit that serves as a heat generation source such as the X-ray generation source 5, in the vicinity of the operation panel 17 having electric components vulnerable to heat, and the like, and may be connected to the first control portion 16 so as to output a high-temperature alarm at a temperature equal to or higher than a predetermined reference value.

The first control portion 16 outputs a control signal to the module group. The control signal referred to here is, for example, a voltage signal defined by a high level or a low level, and in another example, the control signal is a command signal for transmitting and receiving a predetermined command by serial communication. Such a control signal includes a power control signal that controls power supply to each electric unit. By turning off a switch by this power control signal, the power supplied to a specific electric unit can be cut off.

The first control portion 16 may cause the X-ray generation source 5 to start emitting the X-ray, and control to start an operation of the cooling function by the air conditioner 9 in a case where the temperature by the temperature sensor (not illustrated) is equal to or higher than a predetermined value.

Further, the X-ray inspection apparatus 1 includes a second control portion 18. The second control portion 18 controls communication with an external device and the like, and cooperates with the first control portion 16 to transmit and receive an electric signal according to an operating state, an inspection result, or the like of the X-ray inspection apparatus 1 to and from another device and a higher-level management system.

As the control of the X-ray inspection apparatus 1 by the first control portion 16, first, when main power supply is turned on by starting of the apparatus, a control program stored in the storage portion is read into the central processing unit (CPU) and an activation sequence is started. In the activation sequence, an initialization of each electric unit or an output of various sensors such as the humidity sensor and the temperature sensor is taken in to start a warm-up operation. With such a warm-up operation, the operating temperature of the electric unit is stabilized, a change in electric characteristic depending on a temperature change is suppressed, and performance is stabilized. In order to shorten a warm-up time, a warm-up heater with a fan may be provided.

When the warm-up operation is completed, a standby state for an inspection start is set, and the first control portion 16 can start an inspection by an operation of the operation panel 17. When the inspection start operation is performed by the operation panel 17, the X-ray generation source 5 emits X-rays having a predetermined intensity, or an operation start signal is output to a controller (not illustrated) to start the operation of the transport means 7, and a motor as a drive source is operated.

When the transport means 7 starts the operation, the second control portion 18 outputs a signal indicating that the X-ray inspection apparatus 1 can perform an inspection to another device or a higher-level management system.

The X-ray inspection apparatus 1 inspects the inspection object 2 which is sequentially conveyed. A result of the inspection is displayed on the operation panel 17, and is output from the second control portion 18 to another device and a higher-level management system.

Next, for example, in a case where the temperature by the temperature sensor is equal to or higher than a predetermined value, the first control portion 16 turns on the cooling operation of the air conditioner 9 and suppresses an increase in temperature inside the housing 4 when an ambient temperature due to heat generated from the X-ray generation source 5 sharply rises, such as a case where an intensity of the X-rays 3 to be irradiated is strong. Further, the first control portion 16 compares a detected value from the humidity sensor 15 with a preset threshold value. At this time, when the detected humidity of the humidity sensor 15 is higher than the threshold value, that is, in the high humidity state, in order to suppress occurrence of dew condensation, first, the dehumidifying operation of the air conditioner 9 or a dehumidifier that replaces the air conditioner 9 is turned on so as to lower the humidity without lowering the temperature inside the housing 4, and the inside of the housing 4 is dehumidified. The first control portion 16 compares a detected value from the humidity sensor 15 with the threshold value, and stops the dehumidifying operation of the air conditioner 9 and turns on the cooling operation if the detected humidity inside the housing 4 becomes lower than the threshold value. When the dehumidifier is used, the dehumidifier is stopped.

Further, the first control portion 16 outputs a power control signal for cutting off the power supply to the module group, and outputs a high humidity alarm and stops the X-ray inspection so as to turn off the power supply to the module group in which dew condensation is likely to occur while the air conditioner 9 or the dehumidifier is dehumidifying, that is, while the humidity sensor 15 detects the high humidity state.

After stopping the X-ray inspection, the first control portion 16 stops the signal indicating that the inspection is possible for another device before and after. When the high humidity state is lost due to the dehumidifying operation of the air conditioner 9, the power supply is turned on again for the module group in which dew condensation is likely to occur, and the inspection is started.

The first control portion 16 may switch and control the cooling operation and the dehumidifying operation of the air conditioner 9 in consideration of the detected values of the temperature sensor and the humidity sensor 15. For example, when the temperature detected by the temperature sensor is equal to or lower than the operation start temperature of the air conditioner 9 and the humidity detected by the humidity sensor 15 is high, the dehumidifying operation is performed, and in a case where both the temperature and the humidity are high, the cooling operation is performed.

Both the cooling operation and the dehumidifying operation of the air conditioner 9 have a dehumidifying effect since water vapor is liquefied and discharged in a heat exchanger. Therefore, even in a case where the air conditioner 9 does not have the dehumidifying function, based on the detected value of the humidity sensor 15, the first control portion 16 may control the air conditioner 9 so as to suppress dew condensation of the module group, in preference to a condition that the increase in temperature inside the housing 4 requires the cooling operation. In a case where the threshold value is, for example, a relative humidity of 60%, the first control portion 16 monitors the humidity, and turns on the power of the module group when the relative humidity becomes 60% or lower. When the relative humidity exceeds 60%, the power of the module group is controlled to be turned off.

Here, for example, an example in which the X-ray inspection apparatus 1 is installed in a meat processing line at which washing using high-temperature steam is routinely performed will be described.

In a case where a temperature of an atmosphere is 30° C. and a relative humidity is 80%, an absolute humidity is approximately 0.022 kg/kg (DA) from the psychrometric chart. The amount of water vapor in the air at this time corresponds to approximately 22 g/m³. In a case where the temperature of the atmosphere inside the housing 4 is 25° C. and the relative humidity is 60% due to the dehumidifying function of the air conditioner 9, the absolute humidity is approximately 0.012 kg/kg (DA) from the psychrometric chart. The amount of water vapor in the air at this time corresponds to approximately 12 g/m³. Therefore, approximately 10 g/m³ is dehumidified inside the housing 4, and the atmosphere is such that the module group is less likely to cause dew condensation.

Immediately after the meat processing line is stopped, a surface of the housing 4 or the transport means 7 of the X-ray inspection apparatus 1 are washed with high-temperature steam. In addition to fats of attached meats being washed away by the high-temperature steam, the area around the apparatus becomes in a humid state. The meat processing line is left to stand until the meat processing line is restarted, but the housing 4 sucks in the outside air in the humid state from a slight gap of a cover or a door due to a decrease in air pressure due to a decrease in internal temperature. In this manner, the air in the humid state is taken into the housing 4.

When the main power supply of the X-ray inspection apparatus 1 is turned on for restarting, the warm-up operation is started.

During the inspection operation of the X-ray inspection apparatus 1, the X-ray generation source 5 becomes hot and the air conditioner 9 is frequently operated, so that the X-ray inspection apparatus 1 is in a certain dehumidifying state. The amount of heat generated becomes large when the X-ray generation source 5 emits X-rays with an intensity equal to or higher than a predetermined level although the amount of heat depends on the atmosphere in which the X-ray inspection apparatus 1 is installed. Therefore, it is found that the humidity is generally below the threshold value due to continuous dehumidification by the cooling operation of the air conditioner 9. However, if the air in the humid state exists inside the housing 4 immediately after the warm-up operation is completed, the inspection is started, and the like, dew condensation may occur in the locally low-temperature portion, that is, the locally cooled X-ray detection portion 6. During the inspection operation, for example, if the detected humidity exceeds the threshold value, a dew condensation alarm may be issued.

In the example described above, after the main power supply of the X-ray inspection apparatus 1 is turned on and the warm-up operation is completed, the air in the humid state exists around the X-ray detection portion 6, and dew condensation is likely to occur in the vicinity of the Pelche element that locally cools the semiconductor chip of the line sensor unit constituting the X-ray detection portion 6.

Next, an action of the above configuration will be described.

In the X-ray inspection apparatus 1 according to the present embodiment, the cooling means is provided inside the housing 4. In order to protect the electric components from heat, the cooling means sends cold air to the X-ray generation source 5 and the X-ray detection portion 6, for example, and perform cooling by heat transfer by circulating and transporting the cold air inside the housing 4, and promotes heat dissipation from the electric components. Further, in the X-ray inspection apparatus 1, the X-ray detection portion 6 detects X-rays 3 transmitted by irradiating the inspection object 2. The Pelche element as local cooling means is provided in contact with the X-ray detection portion 6 to protect the semiconductor chip of the line sensor unit from heat mainly by heat conduction. That is, the X-ray detection portion 6 is a module group that operates in a cold state. Therefore, it can be said that the X-ray detection portion 6 is a group of modules in which dew condensation is likely to occur due to cooling of the local cooling means, there is a risk of failure due to adhesion of dew condensation water droplets, and electric components that are vulnerable to dew condensation are mounted.

In the related art, if the module group including the Pelche element is operated at the same time in a standby state in which the main power supply of the X-ray inspection apparatus 1 is turned on and an inspection can be started, dew condensation occurs in the module group due to a local temperature difference (or relative humidity), and water droplets adhere to a surface of the electric component, which causes a failure of the semiconductor chip.

In the X-ray inspection apparatus 1 of the present embodiment, a humidity in the vicinity of the module group that is vulnerable to dew condensation inside the housing 4 is monitored by the humidity sensor 15. The first control portion 16 controls the supplying of power to each electric unit, based on a detected value of the humidity sensor 15. If the humidity by the humidity sensor 15 is equal to or higher than a predetermined value, the X-ray detection portion 6 having the module group vulnerable to dew condensation is not turned on. As a result, it is possible to suppress that when the humidity is high, the Pelche element provided in the module group in the X-ray detection portion 6 is not driven, so that the line sensor unit fails due to water droplets adhering to the semiconductor chip of the line sensor unit, for example. In a case where the detected value of the humidity sensor 15 is equal to or lower than the predetermined value, the first control portion 16 performs supplying of power to the module group, that is, the X-ray detection portion 6. Therefore, with the X-ray inspection apparatus 1, dew condensation of the module group integrally provided with the local cooling means can be effectively suppressed.

In consideration of only the semiconductor chip of the line sensor unit constituting the X-ray detection portion 6, when the main power supply of the X-ray inspection apparatus 1 is turned on and the warm-up operation is completed, first, the first control portion 16 monitors the detected value of the humidity sensor 15, and if the humidity is equal to or higher than the threshold value, the inside of the housing 4 is dehumidified by the dehumidifying operation of the dehumidifying means (air conditioner 9), in a state in which only the power supplied to the semiconductor chip and its peripheral circuits is turned off. In a case where the detected value of the humidity sensor 15 falls below the threshold value, for example, when the main power supply is turned on at a state of the initial relative humidity of 90% and dehumidification is performed, and when the internal relative humidity falls below 70% to 60%, the first control portion 16 turns on the power supply to the semiconductor chip and its peripheral circuits. That is, after suppressing the humidity to some extent, the power of the module group that is vulnerable to dew condensation is turned on. As a result, the X-ray inspection apparatus 1 can solve the problem that dew condensation occurs in the module group, especially at the start of the operation. After that, an operation as an apparatus, that is, an inspection will start.

As a result, with the X-ray inspection apparatus 1, it is possible to prevent dew condensation of the electric components due to the cooling of the local cooling means and suppress the failure of the electric components.

In this X-ray inspection apparatus 1, the cooling means includes the air conditioner 9. The including of the air conditioner 9 means that the Pelche element described above is also included as the cooling means. The cooling means is the air conditioner 9 that circulates cold air inside the housing 4 to cool the space inside the housing 4, and is also a cooling unit such as the Pelche element integrally incorporated in a specific module group to locally cool the module group. The humidity sensor 15 may be disposed in the vicinity of the module group in the X-ray detection portion 6, at a position at which the cooling by the cooling unit such as the Pelche element reaches.

The air conditioner 9 has the dehumidifying function. The air conditioner 9 has a refrigeration cycle in which a compressor, a condenser, an expansion valve, an evaporator, and the like are connected by piping to circulate refrigerants, to form a heat exchanger as a general structure. The refrigerant compressed from the compressor into heated steam is sent to the condenser and cooled to become a liquid. This liquid refrigerant evaporates in the evaporator by passing through the expansion valve, and cools the evaporator by taking away the heat of vaporization. By sending air from a blower fan to this evaporator, the heat-exchanged cold air is supplied to the inside of the housing 4.

The dehumidifying function of the air conditioner 9 utilizes the dew condensation in the evaporator at this low temperature. That is, the evaporator is cooled up to a dew point temperature with respect to the relative humidity of the air inside the housing 4 sent by the blower fan, and water vapor is discharged to the outside of the apparatus as dew water and removed.

The purpose of dehumidification is to remove moisture in the atmosphere, and cold air is blown out from the evaporator in the same manner as cooling. In a case where only dehumidification is required and cooling is not required, the blown air is reheated. As a result, the relative humidity can be significantly reduced without changing the temperature inside the housing 4, and the atmosphere inside the housing 4 can be made less likely to cause dew condensation. Further, in the X-ray inspection apparatus 1, the X-ray generation source 5 is desired to be warmed. Meanwhile, the X-ray generation source 5 is not desired to be heated up to a high temperature. The module group having the semiconductor chip has different demands for warming but cooling. Therefore, the operating temperature of each electric unit inside is stabilized by the warm-up operation at the start of the operation, and the air conditioner 9 performs not only the dehumidifying operation but also the cooling operation. The X-ray inspection apparatus 1 can cool the inside of the housing 4 and dehumidify the inside of the housing 4, by using the function of the air conditioner 9.

Further, by using the air conditioner 9 that circulates cold air inside the housing 4 of the X-ray inspection apparatus 1 in combination with the cooling means such as the Pelche element that locally cools the contact surface, the cooling and the dehumidification inside the housing 4 and the cooling of the module group that is vulnerable to dew condensation can be respectively achieved in a well-balanced manner.

With this X-ray inspection apparatus 1, in a case where the temperature detected by the temperature sensor becomes equal to or higher than the predetermined value, the operation of the cooling function by the air conditioner 9 is started. That is, for example, the X-ray generation source 5 or the like is heated by energization, and then the cooling function or the dehumidifying function of the air conditioner 9 is turned on. As a result, it becomes possible to maintain the temperature inside the housing 4 stabilized by the warm-up operation, a performance change of the electric unit due to a temperature change can be suppressed, and the performance of the X-ray inspection apparatus is stabilized. The X-ray generation source 5 has the tank, and the tank is filled with cooling oil to accommodate the X-ray source. The tank cooling portion provided on the upper surface of the tank has the heat dissipation fin covered with the cover. At a start of the operation, the X-ray inspection apparatus 1 requires the warm-up operation so as to bring the X-ray source to a predetermined temperature. However, in order to avoid becoming too warm, that is, a high-temperature state, cooling by the air conditioner 9 is required. As a result, the warm-up operation can be performed and the inside of the housing 4 can be dehumidified.

Therefore, with the X-ray inspection apparatus 1 according to the present embodiment, it is possible to prevent dew condensation of the electric components while cooling the inside of the housing 4 and suppress a failure of the electric components.

### DESCRIPTION OF REFERENCE NUMERALS AND SIGNS

1 . . . X-ray inspection apparatus
2 . . . Inspection object
3 . . . X-ray
4 . . . Housing
5 . . . X-ray generation source (non-condensation module group)
6 . . . X-ray detection portion (module group)
9 . . . Air conditioner (dehumidifying means, cooling means)
15 . . . Humidity sensor
16 . . . First control portion (control portion)

What is claimed is:

1. An X-ray inspection apparatus that inspects an inspection object by irradiating the inspection object with X-rays and detecting transmitted X-rays, the apparatus comprising:
a housing;
cooling means for cooling an inside of the housing;
a module group which is disposed inside the housing and in which an electric component that is vulnerable to dew condensation caused by cooling of the cooling means is mounted;
a humidity sensor that monitors a humidity inside the housing; and
a control portion that performs supplying of power to the module group when the humidity monitored by the humidity sensor is equal to or lower than a predetermined value.

2. The X-ray inspection apparatus according to claim 1,
wherein after the X-ray inspection apparatus is activated, the control portion starts the supplying of power to the module group, under a condition that the humidity monitored by the humidity sensor is equal to or lower than the predetermined value.

3. The X-ray inspection apparatus according to claim 1, further comprising:
dehumidifying means for dehumidifying the inside of the housing,
wherein when the humidity monitored by the humidity sensor is equal to or higher than the predetermined value, the control portion causes the dehumidifying means to dehumidify the inside of the housing, and stops the supplying of power to the module group.

4. The X-ray inspection apparatus according to claim 2, further comprising:
dehumidifying means for dehumidifying the inside of the housing,
wherein when the humidity monitored by the humidity sensor is equal to or higher than the predetermined value, the control portion causes the dehumidifying means to dehumidify the inside of the housing, and stops the supplying of power to the module group.

5. The X-ray inspection apparatus according to claim 3,
wherein the control portion further issues an alarm when the humidity monitored by the humidity sensor is equal to or higher than the predetermined value.

6. The X-ray inspection apparatus according to claim 4,
wherein the control portion further issues an alarm when the humidity monitored by the humidity sensor is equal to or higher than the predetermined value.

7. The X-ray inspection apparatus according to claim 3,
wherein the cooling means includes an air conditioner, and
the air conditioner has a dehumidifying function as the dehumidifying means.

8. The X-ray inspection apparatus according to claim 4,
wherein the cooling means includes an air conditioner, and
the air conditioner has a dehumidifying function as the dehumidifying means.

9. The X-ray inspection apparatus according to claim 7,
wherein inside the housing, a non-condensation module group that is not affected by dew condensation and different from the module group is provided, and a temperature sensor that monitors a temperature inside the housing is provided, and
the control portion supplies power to the non-condensation module group, and then starts an operation of a cooling function by the air conditioner in a case where the temperature monitored by the temperature sensor is equal to or higher than the predetermined value.

10. The X-ray inspection apparatus according to claim 8,
wherein inside the housing, a non-condensation module group that is not affected by dew condensation and different from the module group is provided, and a temperature sensor that monitors a temperature inside the housing is provided, and
the control portion supplies power to the non-condensation module group, and then starts an operation of a cooling function by the air conditioner in a case where the temperature monitored by the temperature sensor is equal to or higher than the predetermined value.

11. The X-ray inspection apparatus according to claim 9,
wherein the control portion switches between a cooling operation and a dehumidifying operation of the air conditioner, based on a detected value of the temperature sensor and a detected value of the humidity sensor.

12. The X-ray inspection apparatus according to claim 10,
wherein the control portion switches between a cooling operation and a dehumidifying operation of the air conditioner, based on a detected value of the temperature sensor and a detected value of the humidity sensor.
